(19) United States
(12) Patent Application Publication
(10) Pub. No.: US 2018/0156792 A1
(43) Pub. Date: Jun. 7, 2018

(54) SAMPLING PORTION FOR A TEST DEVICE

(71) Applicant: Ellume Pty Ltd., East Brisbane, Queensland (AU)

(72) Inventors: Sean Andrew PARSONS, East Brisbane (AU); Richard Francis DAY, Great Cambourne (UK); Lucie BROWNING, Cambridge (UK)

(21) Appl. No.: 14/786,963

(22) PCT Filed: Apr. 28, 2014
(86) PCT No.: PCT/AU2014/000470

§ 371 Date: Oct. 23, 2015

(30) Foreign Application Priority Data
Apr. 26, 2013 (AU) 2013901449

(51) Int. Cl. G01N 33/558; B01L 3/00

(52) U.S. Cl. CPC G01N 33/558; B01L 3/5023; B01L 3/5055; B01L 2200/04; B01L 2300/027; B01L 2400/0406; B01L 2300/047; B01L 2300/0672; B01L 2300/0816; B01L 2300/0883; B01L 2300/161; B01L 2300/044

(57) ABSTRACT

A test device is disclosed for determining the presence or absence of a biological entity in a biological sample from a human or animal body, the test device comprising a sampling portion, the sampling portion comprising absorbent material forming a liquid flow path having a series of bends. The flow path provides a circuitous route for fluid such as buffer solution to flow within the sampling portion, improving combining of fluid with a sample received by the sampling portion.

### CROSS-REFERENCE TO RELATED APPLICATIONS

The present application claims priority from Australian provisional patent application no. 2013901449 filed on Apr. 26, 2013, the contents of which are incorporated herein by reference in their entirety.

### FIELD

The present disclosure relates to test devices such as immunoassays.

### BACKGROUND

Devices for testing biological samples routinely, employ a lateral flow medium that allows transfer of a sample through the device as part of the testing process.

For example, immunoassays are commonly used to test for the presence or absence of an antigen in a biological sample. The sample, such as urine, blood or mucus, is supplied to a sampling portion of a lateral, flow medium and flows by capillary action through a label-holding substance which contains a soluble and labelled antibody specific to a particular antigen. If that particular antigen is present in the sample, an antigen-antibody (labelled) complex is formed which then continues to permeate by capillary action through the device to a test site where the complex is captured by a second antibody attached to the test site. This results in an increase in the density of captured antigen-antibody (labelled) complexes at the test site which can result in a visible mark (usually a line) on the test site being formed to indicate the presence of the antigen in the sample.

It can be desirable to increase the fluidity of the biological sample to improve flow of the sample through a test device. Increasing fluidity may be particularly important where the sample is relatively viscous. To increase fluidity, a liquid such as a buffer solution, may be combined with the sample.

The liquid may be introduced from an external source by placing the liquid onto the test device, e.g. using a dropper such as a pipette, etc. The liquid may be combined with the sample prior to, or after, placement on the test device. As an alternative to dropping the liquid, onto the test device, a liquid reservoir can be included with the test device that is releasable to allow liquid contained in the reservoir to combine with the sample.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

According to one aspect, the present disclosure provides a test device including a sampling portion, the sampling portion including absorbent material forming a sample flow path having a series of bends.

The sampling portion may have a sample receiving surface adapted to receive a sample, deposited thereon. The arrangement may be such that sample can be placed anywhere on the sampling portion including directly on bends of the flow path. The receiving surface may be formed by the absorbent material such that sample deposited thereon can be drawn into the sample flow path.

The test device may comprise a liquid transfer portion that is connected or is configured to be connected to the sampling portion such that liquid is transferable from the liquid transfer portion to the flow path.

Liquid may travel through the liquid transfer portion, by capillary action, and the liquid, and a liquid and sample combination, may travel through the sample flow path by capillary action. The liquid transfer portion and the sampling portion may be formed from a unitary lateral flow medium or may be provided by separate lateral flow mediums that are connected or configured to be connected together. The liquid transfer portion and the sampling portion may be connected together prior to receipt of the sample or connected together only after receipt of the sample, e.g. as part of a sample processing step. In one embodiment, connecting together of the liquid transfer portion and the sampling portion, may cause liquid to be released from the reservoir.

The lateral flow medium may comprise nitrocellulose, glass fibre, paper, or other suitable wicking material that enables transfer of the liquid and sample therethrough.

The device may be configured such, that liquid transferred to the sample flow path combines with the received sample. The series of bends may ensure that liquid that, is transferred to the sampling portion is forced to follow a route through the sampling portion that will necessarily navigate past or through, substantially any position on the sampling portion at which the sample is received. The flow path may be considered to take a substantially circuitous, winding and/or meandering route though the sampling portion. The flow path may traverse substantially backwards and forwards and/or side to side across the sampling portion. The flow path may include at least two bends, at least three bends or four or more bends.

The width of the flow path at any point along the flow path may be substantially narrower than the width of the area over which the path taken by the fluid flow path extends, e.g. narrower than the width of the sampling portion. For example, the flow path may have a maximum width that is less than, half or less than one third of less than, one quarter of the maximum width of the area over which the flow path extends. Thus, while the flow path may be relatively narrow at any point, the area over which the flow path extends, i.e. the area covered by the flow path, may be relatively large. By providing a relatively narrow, bent flow path, rather than a flow path that has a width extending across the entire sampling portion, liquid may be prevented from travelling through the flow path in such a way that it does not combine with the sample. In essence, the possibility for liquid to find a route through the flow path that circumvents or bypasses the deposited sample can be reduced or eliminated.

Liquid may be combined with the sample for the purpose of increasing its fluidity. This may allow the sample to be subjected to further processing though the test device more easily. For example, it may enable, or at least improve, transfer of the sample through the sample flow path and other portions of the device.

Nonetheless, liquid may be combined with the sample for additional, or alternative reasons. For example, it may be desired to chemically treat the sample and as such, the liquid may have a particular chemical composition that induces a chemical reaction and/modifies physical properties of the sample, other than fluidity, when combined with the sample.

The test device may comprise a liquid reservoir connected to the liquid transfer portion. Once a sample has been received by the sampling portion, the liquid may be released from the reservoir and may transfer automatically to the sampling portion. Alternatively, liquid may be delivered to the liquid transfer portion by other means. For example, a dropper such as a pipette may be used to deposit liquid on the liquid transfer portion, or the liquid transfer portion may be dipped in a receptacle containing the liquid.

The liquid reservoir may be a sealed reservoir containing the liquid and which is breakable and/or has a removable portion so that the liquid can be released. For example, the reservoir may take the form of a capsule, bubble or blister containing the liquid, or container having at least one thin wall, which is capable of breaking or bursting to release the liquid. The reservoir may have a weakened part to facilitate easier breaking or bursting of the reservoir, and this may be at a predetermined position so that released liquid is distributed to an appropriate part of the device. An element may be provided in the device that is actuatable to break, or burst the reservoir, which element may comprise a sharp point, for example.

The test device may comprise a test portion that is connected to or configured to be connected to the sampling portion. The test device may be configured such that liquid that combines with the sample in the sampling portion is transferred to the test portion, e.g. by capillary action. The test portion may be configured to test for the presence or absence of a biological entity in the sample using immunochromatography or other techniques. The sampling portion may be connected directly to the test portion or connected to the test portion via another liquid transfer portion. The liquid transfer portion(s), the sample flow path and the test portion may be provided by a unitary lateral flow medium or may be provided by separate lateral flow mediums that are connected or configured to be connected together. The separate lateral flow mediums may be connected together prior to receipt of the sample or connected together only after receipt of the sample, e.g. as part of a sample processing step.

The series of bends of the sample flow path may include a plurality of bends of at least 90 degrees. In one embodiment, the series of bends may include a plurality of bends of approximately 180 degrees, in one embodiment, the series of bends may include a plurality of bends of approximately 90 degrees and a plurality of bends of approximately 180 degrees. The bends may be configured in some embodiments such that the flow path effectively doubles back on itself. Therefore different, portions of the flow path may travel in substantially opposite directions.

The sampling portion may be flat and the series of bends of the flow path may lie in a single plane only. The direction of the flow path as it travels through, the sampling portion may therefore change in two dimensions only. However, in alternative embodiments the sampling portion may have a more three dimensional shape and the direction of the flow path may change in three dimensions.

The flow path may have a substantially sinusoidal shape or a substantially square or rectangular wave shape. The series of bends may include one or more bends that are curved and/or one or more bends that are sharp or angled bends. In some embodiments, curved bends may be preferable to prevent the formation of corner areas of the flow path where the liquid may pool and/or be less likely to pass through. By providing curved bends, the flow path may maintain the same width along its entire length, improving the predictability of liquid flow.

The flow path may comprise a plurality of substantially straight sections connected to each other via one or more bend sections. A plurality of the substantially straight sections may be substantially parallel, to each other and adjacent/substantially straight sections may be separated from each other by a gap. The gap may be from about 0.5 mm to 5 mm, e.g. from 1 mm to 3 mm. In one embodiment, the gap may be about 2 mm.

Selecting the appropriate size of gaps between adjacent sections of the sample flow path may be a balance between (i) having small gaps so that the flow path provides a relatively complete region of the sampling portion, e.g. so that sample is more likely to be absorbed into the flow path rather than falling through or sitting across gaps in the flow path; and (ii) having large gaps that prevent liquid jumping between adjacent sections of the flow path in such a manner that the liquid could bypass a location at which the sample has been received. Within these confines, the appropriate size of the gaps may vary depending on the viscosity of the sample to be tested and the properties of the medium forming the flow path.

The flow path may be defined by opposing outer edges of material. In the same plane or planes as the series of bends of the flow path, gaps between adjacent sections of the flow path may be absent of any material. However, alternatively, the gaps between adjacent sections of the sample flow path may be partially or entirely filled with material. For example, the flow path may be defined by a first material surrounded by a second material, the second material being less absorbent than the first material. The second material may provide a liquid repellent barrier that surrounds the first material. As an example, a wax printing technique may be employed to form the flow path in which hot wax is applied to a surface of absorbent material and penetrates to define hydrophobic barriers that define the flow path.

The flow path of the sampling portion, including the series of bends may be provided over an area having a size that provides a suitable target region for deposition of the particular type of a sample under test. In some embodiments, the area may be greater than 10 cm² and in other embodiments the area may be less than 10 cm² e.g., less than 8 cm², less than 6 cm² or otherwise. Similarly, the maximum dimension of the area over which the sampling portion is provided, including the series of bends, may be greater than 5 cm in some embodiments, while in other embodiments the maximum dimension may be less than 5 cm. e.g., less than 4 cm or less than 3 cm, for example.

The sampling portion may be adjustably conformable to a part of a human or animal body for receiving a biological sample directly from the body. In this regard, the test device may be configured substantially in accordance with a test device as disclosed in PCT publication no. WO 2011/091473 A1, the content of which is incorporated herein by reference. The sampling portion may comprise flexible material that is sufficiently supple to bend or fold freely or repeatedly in order to conform to a variety of different shapes of body parts to receive a biological sample. The flexible material may be bent or folded repeatedly without being substantially damaged, cosmetically and/or and functionally. In one embodiment, the device may take, generally, a butterfly configuration. The device may include two flexible wings at least partially forming the sampling portion, and a central housing (spine) located between the two wings. The wings may be relatively pivotable or flexible about the housing.

The device, may detect the presence or absence of one or more specific biological entities, such as antigens. The antigens may be found in common respiratory viruses including but not limited to Influenza A (including the H1N1 virus subtype), Influenza B, Respiratory Synctial Virus, parainfluenza viruses, adenoviruses, rhinoviruses, coronaviruses, coxsackie viruses, HIV viruses and/or enteroviruses. The device may also detect specific biological antigens found in bacteria, fungi, protozoa, Helminths, Mycoplasma and prions. The device may also be capable of detecting specific proteins produced by the human or animal body; including but not limited to immunoglobulin, hormone molecules, inflammatory or malignant proteins. The test portion of the device may comprise a plurality of different test zones so that the presence or absence of different biological entities such as antigens can be tested simultaneously.

The test device may comprise a cover layer and/or a backing layer. The cover layer may be attached to, and extend over, one side of the sampling portion. A hole or absorbent portion of the cover layer may be provided over the sampling portion to allow transfer of the sample through the cover layer to the flow path. The flow path including its series of bends, may thus be provided underneath a target region in the cover layer at which, sample is to be received. Where the cover layer is at least partially absorbent, the cover layer may be considered a dressing layer. The backing layer may provide a hydrophobic or liquid repellent surface on an opposite side of the sampling portion. The backing layer may ensure that the sample, received by the sampling portion does not leak from the sampling portion, e.g., onto a hand or other surface, and is instead directed through the flow path of the sampling portion.

A housing may be provided in the device and arranged to at least partially enclose and/or protect one or more components of the device. For example, the housing may enclose at least partially the test portion of the device, the liquid reservoir and/or other elements discussed herein. The housing may be substantially rigid and may prevent or reduce the likelihood of damage to the test portion, liquid reservoir and/or other elements enclosed at least partially enclosed therein. When the housing at least partially encloses the test portion, the housing may include one or more openings or transparent portions to permit observation of indicia showing the results of testing.

In one embodiment, the device may comprise one or more lateral flow test strips, in the form of relatively rigid elongate layered strips of the type commonly used for pregnancy testing, etc. The lateral flow test strips may comprise the test portion. The sampling portion may be integrated into the test strip or may be provided separately from the test strip and connected to the test strip by a liquid transfer portion.

The test portion of the device may be provided with antigens or antibodies to allow testing for the presence of one or more biological, entities using existing principles of lateral flow immunochromatography. One or more label-holding areas, e.g. coloured label-holding areas containing specific antibodies bound to light visible molecules, may be provided in the test portion. The label-holding areas may be located at the edge or adjacent the edge of the test portion at the boundary between the test portion and the sampling portion, for example. The sample received by the sampling portion may travel via capillary action through the sampling portion and into the test portion where it mixes with the label-holding areas and may form antigen-antibody (labelled) complexes. The test zones may comprise stripes (lines); crosses, squares or other shaped regions of the test portion that have been impregnated with, antibodies or antigens. Depending, upon the biological antigens present in the sample, and the antibodies or antigens at the label-holding areas and the test zones, the sample may become bound at one or more of the test zones, causing a colour change at the test zones. The change in colour may be observable by a user and indicative of the presence or absence of a specific biological entity in the sample, such as, but not limited to, influenza A or influenza B. In alternative embodiments, however, an electronic reader may be provided to analyse changes at the test portion and results of testing may be presented to the user through an electronic display, e.g. an LCD or LED display, etc.

Although the device may use principles of immunochromatography, it is conceived, however, that alternative means of testing could be incorporated into the device.

The device may provide a rapid diagnosis test device, permitting testing in less than one hour, less than 30 minutes, less than 10 minutes, less than 5 minutes, or less than 2 minutes, for example. The device may be disposable, configured for single-use only. The device may be provided in sterile packaging prior to use. The device may provide a means for entirely non-invasive testing for the presence or absence of one or more biological entities. The device may be used for testing in the veterinary field as well as in the field of human medicine. The device may provide a home use or point-of-care test device.

### BRIEF DESCRIPTION OF DRAWINGS

By way of example only, embodiments are now described with reference to the accompanying drawings, in which:

FIG. 1a shows a sampling portion of a test device according to a first embodiment of the present disclosure and FIGS. 1b and 1e show variations of the sampling portion of FIG. 1a.

FIGS. 2a to 2e show sampling portions according to further embodiments of the present disclosure.

FIGS. 3a and 3b show a comparative example of a sampling portion to which a sample is applied and through which buffer solution is caused to flow;

FIGS. 4a and 4b show an embodiment of a sampling portion according to the present disclosure to which a sample is applied and through which buffer solution is caused to flow;

FIG. 5 shows a schematic view of a device according to an embodiment of the present disclosure;

FIGS. 6a and 6b show opposing side views of the device of FIG. 5, and FIG. 6c shows an end view of the device of FIG. 5;

FIG. 7 shows an exploded view of the device of FIG. 5;

FIGS. 8a and 8b show bottom and top views respectively of a spine of the device of FIG. 5;

FIG. 9 shows a partial cross-sectional view of the spine of the device of FIG. 5;

FIGS. 10a to 10c show oblique cross-sectional views of the device of FIG. 5 with a slider in different actuation positions;

FIG. 11 shows a schematic plan view of a test strip for use in the device of FIG. 5;

FIG. 12 shows a schematic plan view of a test strip of a test device according to another, embodiment of the present disclosure; and

FIG. 13 shows a cross-sectional side view of the test device including the strip of FIG. 12.

### DESCRIPTION OF EMBODIMENTS

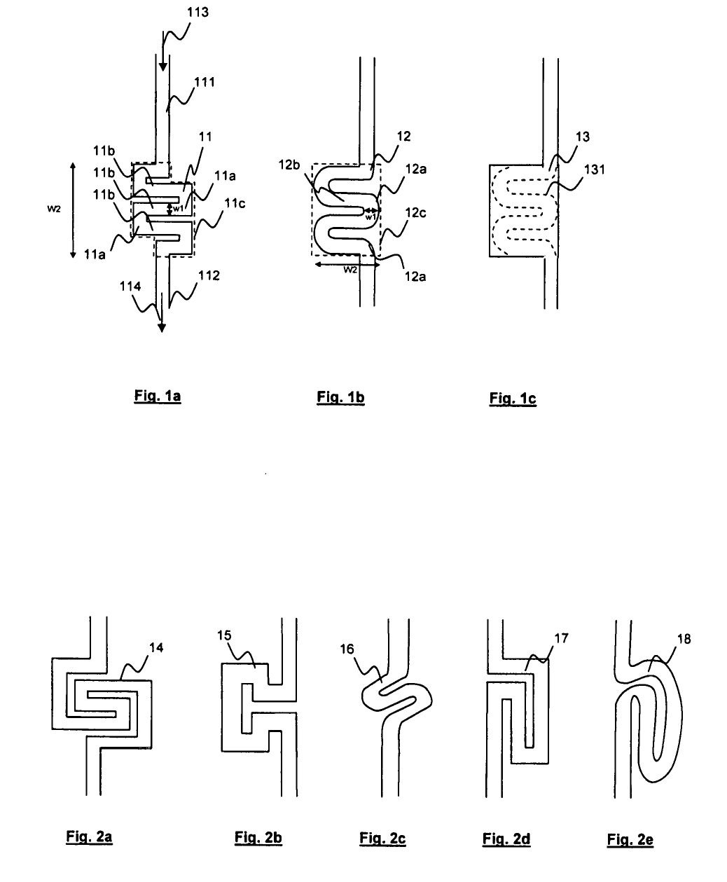

A portion of a test device according to a first embodiment of the present disclosure is now discussed with reference to FIG. 1a. The test device comprises a substantially flat, lateral flow medium that defines a first liquid transfer portion (first arm 111), a sampling portion 11, and a second liquid transfer portion (second arm 112).

The sampling portion is adapted to receive a biological sample, such as mucus, blood or urine, that is deposited on a top surface of the sampling portion. The sample can be at least partially absorbed into the sampling portion. The first arm 111 is configured to deliver liquid such as buffer solution, as represented by arrow 113, to the sampling portion where it combines with the sample and causes dilution of the sample. The second arm 112 is configured to deliver the combined sample and liquid, as represented by arrow 114, to a test portion of the test device for further processing.

The first and second arms 111, 112 define substantially straight flow paths either side of the sampling portion. In alternative embodiments, the first and second arms may define curved or bent flow paths; however, they may still extend along a reasonably direct route to connect to the sample flow path. The sampling portion 11 provides a sample flow path that takes a meandering, circuitous route from one end of the sampling portion 11 to another. The sample flow path has a series of bends 11a so that the sample flow path effectively doubles back on itself. The sample flow path includes bends 11a of approximately 180 degrees, for example.

In this embodiment, the bends 11a of the sample flow path connect substantially parallel, substantially straight sections 11b of the sample flow path together, providing the sample flow path with a substantially square wave shape. By taking a meandering, circuitous route, between the first and second arms 111, 112, the sample flow path extends across a relatively wide area, an area that provides a target region for receipt of a sample, as identified by broken line 11c. The target region 11c is sufficiently large to allow relatively straightforward deposition of sample thereon.

While the flow path of the sampling portion 11 has a substantially square wave shape, in an alternative embodiment, as shown in FIG. 1b, the flow path of the sampling portion 12 may have a substantially sinusoidal shape. By employing smooth, curved, bends instead of sharp corner sections, the flow path can have substantially the same width as it extends throughout the sampling portion 12, reducing or eliminating the formation of corner areas where liquid may pool or be less likely to pass through. The flow path of the sampling portion 12 of FIG. 1b also differs from the flow path of the sampling portion 11 of FIG. 1a by the position in the sampling portion at which the bending of the flow path starts and ends. As shown in FIG. 1b, the first and last bends 12a are located at a lateral side of the sampling portion 12, rather than at a central location. This provides a target region 12c, over which the flow path extends, with an even shape. The target region 12c may take a substantially rectangular or square shape, for example.

The flow path is not limited, however, to any particular meandering pattern or shape. Examples of flow paths of sampling portions 14-18 according to other embodiments of the present disclosure are represented in FIGS. 2a to 2e. In each case, the flow path includes bends of at least 90 degrees or 180 degrees and there are sections of the flow path that travel in opposite directions to other sections of the flow path. The flow paths of sampling portions 14, 15 and 17 illustrated in FIGS. 2a, 2b and 2d can be considered to have shapes corresponding to single repeated units of Greek fret or Greek key designs (e.g., meandros shapes). While similar, the flow paths of sampling portions 16, 18 illustrated in FIGS. 2c and 2e have curved bends so that corner areas are not formed where liquid may pool or where liquid may be less likely to pass through, as discussed above.

In each of the sampling portions represented in FIGS. 1a to 2e, adjacent sections of the flow path are relatively close to each other, thus avoiding the formation of large gaps in the sampling portion across which no portion of the flow path extends. Adjacent, e.g. parallel, sections of the flow path may be separated from each other by a gap that is from about 0.5 mm to 5 mm, e.g. from 1 mm to 3 mm. In one embodiment, the gap may be about 2 mm.

The appropriate size of gaps between adjacent sections of the sample flow path is selected in these embodiments as a balance between (i) having small gaps so that the flow path provides a relatively complete target region of the sampling portion, e.g. so that sample is more likely to be absorbed into the flow path rather than falling through or sitting across gaps in the flow path; and (ii) having large gaps that prevent liquid jumping between adjacent sections of the flow path in such a manner that the liquid could bypass a location at which the sample has been received. Within these confines, the appropriate size of the gaps is varied depending on the viscosity of the sample to be tested and the properties of the medium forming the flow path.

As shown in FIGS. 1a and 1b, for example, the sample flow path can be defined by opposing outer edges of absorbent material. In the same plane as the flow path, gaps between adjacent sections of the flow path are absent of any material. However, alternatively, gaps between adjacent sections of the sample flow path may be partially or entirely filled with material. For example, the flow path can be defined by a first material surrounded by a second material, the second material being less absorbent than the first material. The second material can provide, a liquid repellent barrier that surrounds the first material. As an example, in the embodiment shown in FIG. 1c, a wax printing technique has been employed to form a flow path in a sampling portion 13. Hot wax has been applied, to a surface of the absorbent material of the sampling portion 13 and has penetrated through the absorbent material to provide hydrophobic barriers 131 that define the flow path.

As seen in FIGS. 1a and 1b, a width w1 at any point along the sample flow path (i.e. the dimension of the flow path perpendicular to the direction of the flow path at any particular point) is substantially narrower than the width w2 of the target, region, 11c, 12c, over which the flow path extends. Considered another way, a width w1 at any point along the sample flow path is substantially narrower than the dimension w2 of the target area 11c, 12c in a direction parallel to the width, w1 of the flow path. In the embodiments shown in FIGS. 1a and 1b, for example, the flow path of the sampling portion has a maximum width w1 that is less than a quarter of the width w2 of the target region 11c, 12c. In these embodiments, the width w1 of the flow path is about 0.5 cm and the width w2 of the target region over which the flow path extends is about 2.5 cm.

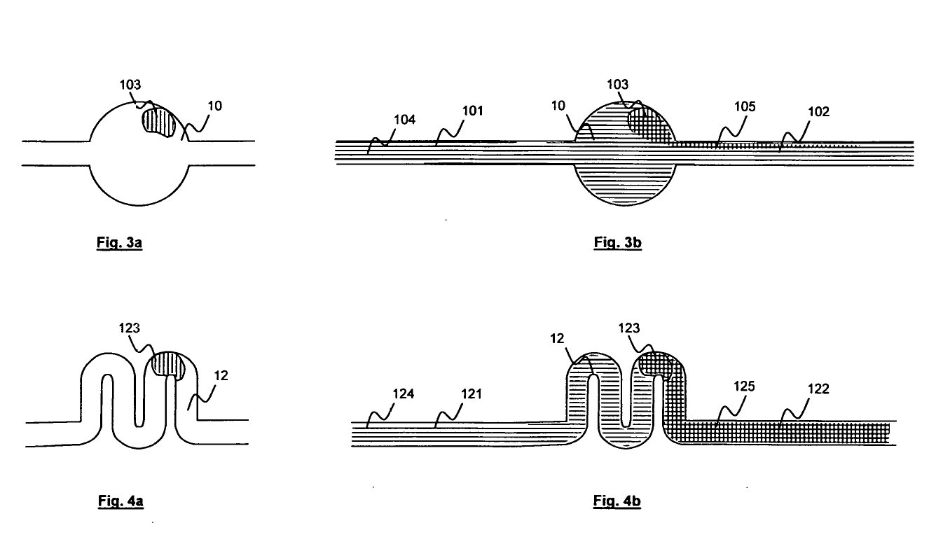

By providing a relatively narrow, bent, flow path, rather than a flow path that has a width extending across the entire sampling portion (e.g., where w1=w2), liquid may be prevented from travelling through the flow path, in such a way that it does not combine with sample that has been received by the sampling portion. The possibility for liquid to find a route through the flow path that circumvents or bypasses deposited sample can be reduced or eliminated. This is now discussed in more detail with reference to FIGS. 3a to 4b.

FIG. 3a shows a comparative example sampling portion 10 that is formed from a single substantially circular piece of absorbent material. One of many positions at which a sample 103 could be deposited on the sampling portion 10 is shown in FIG. 3a. The sample 103 is represented in FIGS. 3a and 3b using vertical lines.

FIG. 3b shows the same sampling portion 10, through which a liquid, in particular a buffer solution 104, has passed. The buffer solution 104 is represented in FIG. 3b using horizontal lines. The buffer solution has transferred through the first arm 101, e.g. from a reservoir connected to the first arm 101, through the sampling portion 10, and though the second arm 102. At the position of the sampling portion 10 at which the sample 103 has been deposited, some engagement of the solution 104 and the sample 103 has taken place such as to form a sample and solution combination 105. The sample and solution combination 105 is represented in FIG. 3b using crossed horizontal and vertical lines.

As can be seen in FIG. 3b, while some of the solution 104 has combined with the sample 103, a substantial portion of the solution 104 has bypassed the sample 103 altogether. While some diffusion of the solution 104 across the entire sampling portion 10 has taken place, the bulk of the solution 104 has travelled along a straight path between the first and second arms 101, 102, without engaging the sample 103. Accordingly, combining of the sample 103 and solution 104 is relatively minimal in this example. Indeed, the sample and solution combination 105 that has travelled through the second arm 102, and which would desirably be subject to further processing, may be too small in volume to achieve any accurate testing results.

FIG. 4a shows a sampling portion 12 according to the present disclosure, which defines it substantially sinusoidal flow path. The flow path extends over an area that is similar in size to the area over which the sampling portion 10 of FIG. 3a extends. One of many positions at which a sample 123 can he deposited on the sampling portion 12 is shown in FIG. 4a. To enable direct comparison, the position of the sample 123 shown in FIG. 4a correlates to the position of the sample 103 shown in FIG. 3a. The sample 123 is also represented in FIGS. 4a and 4b using vertical lines.

FIG. 4b shows the same sampling portion 12, through which a liquid, in particular a buffer solution 124, has passed. The buffer solution 124 is also represented in FIG. 4b using horizontal lines. The buffer solution 124 has transferred through the first arm 121, e.g., from a reservoir connected to the first arm 121, through the sampling portion 12, and though the second arm 122. At the position of the sampling portion 12 at which the sample 123 has been deposited, engagement of the solution 124 and the sample 123 has taken place such as to form a sample and solution combination 125. The sample and solution combination 125 is also represented in FIG. 4b using crossed horizontal and vertical lines.

As can he seen in FIG. 4b, due to the configuration of the flow path through the sampling portion 12, substantially all of the solution 124 has combined with the sample 123. This is because there is substantially no route through the flow path for the solution 124 to take that does not pass proximate to the sample 123. Accordingly, combining of the sample and solution is relatively high in this example. The sample and solution combination 125 that has travelled through the second aim 122, which can be subjected to further processing, is sufficient in volume to achieve accurate testing results.

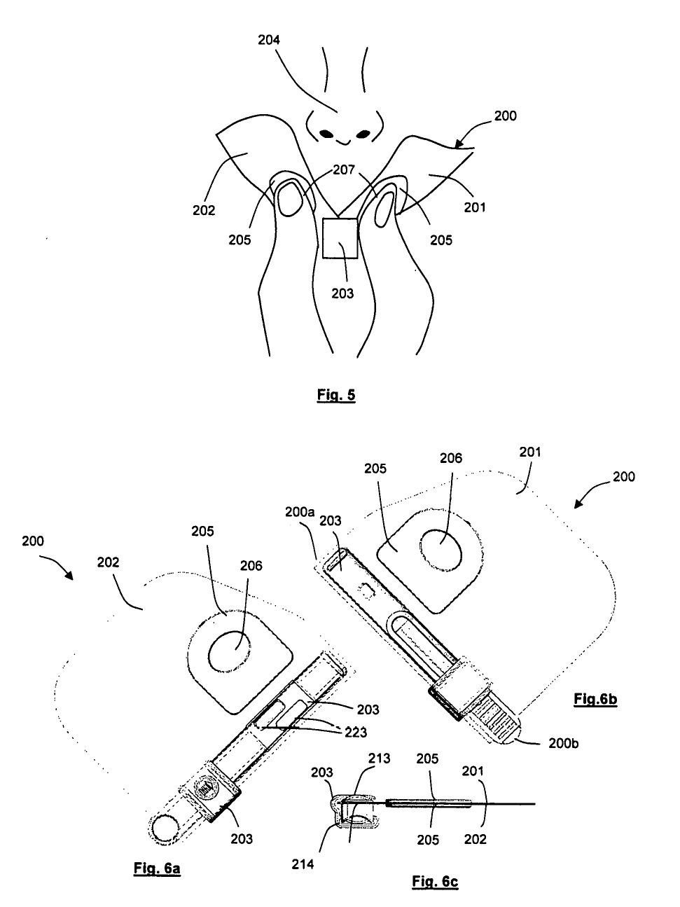

An embodiment of a test device 200 according to the present disclosure is now discussed with reference to FIGS. 5 to 7. The test device 200 is configured in accordance with a test device discussed in PCT publication no. WO 2011/091473 A1, the content of which is incorporated herein by reference. However, in accordance with the present disclosure, the test device has a modified sampling portion that provides a flow path having a series of bends, more particularly a circuitous, meandering flow path.

The device 200 may be considered to take, generally, a butterfly shape, due to the inclusion in the device of two wings 201, 202, provided by two substantially flat and flexible sampling elements, and a spine 203, provided by an elongate central body, the wings 201, 202 extending from, and being relatively pivotable about, the spine 203. The wings 201, 202 are designed to have a sufficiently large surface area, and to be sufficiently pliable, to flex around a person's nose 204, permitting the person to deposit a nasal mucus sample in a region between the two wings 201, 202, using a nose blowing technique. A simplified drawing of the device 200, with the wings 201, 202 in an open configuration, showing how the device 200 may be brought into a position with a nose 204, is provided in FIG. 5. A more detailed drawing of the device 200, with wings 201, 202 in a closed position, e.g., prior to use of the device, or after receipt of the sample, is shown in FIGS. 6a to 6c. As can be seen in these Figures, on the outside of each wing 201, 202, a respective finger locator is provided. Each finger locater includes a pad 205 with a hole 206, for receiving a finger or thumb tip 207. By placing the tips 207 of their thumb and forefinger (or other fingers) in the hole 206 of each locator, the user will generally position the device 200 correctly when it is brought into contact with their nose 204 for nose blowing, so that a nasal sample is received at a targeted location of the device 200. Although this device 200 is configured to obtain and test nasal discharge, e.g., mucus, in alternative embodiments, the device may be configured to obtain and test other biological samples, such as blood, serum, plasma, saliva, sputum, urine, ocular fluid, tears, semen, vaginal discharge, ear secretions, perspiration, mucus, stool, and/or amniotic, spinal, wound, or abscess fluid.

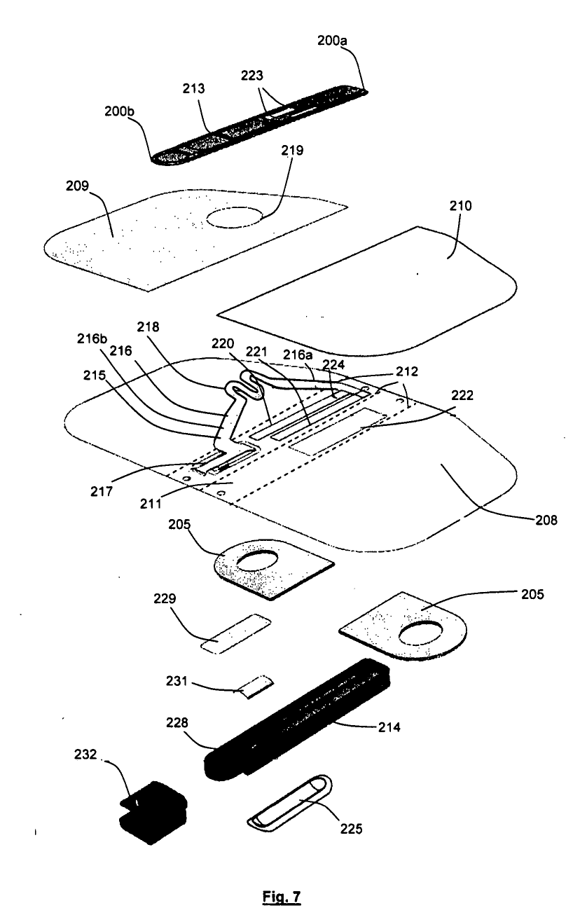

FIG. 7 shows an exploded view of the device 200, allowing the various components of the device 200 to be seen in more detail. The two wings 201, 202 are formed from a waterproof backing layer 208 and respective first and second inner layers 209, 210. The backing layer 208 may be formed of plastic, e.g. a polyester sheet. The backing layer 208 is configured to be folded at a central fold region 211 along three fold lines 212, which region 211, when folded, is sandwiched between a top plate 213 and a main body 214 of the spine 203 (see FIG. 6c, for example). The first and second inner layers 209, 210 are mounted on the inner surface of the backing layer 208 at respective sides of the fold region 211. Between the first inner layer 209 and the backing layer 208, an absorbent pad 215 is provided. The pad 215 provides a lateral flow medium (capillary membrane) and is substantially flexible. In this embodiment, the pad 215 comprises a substantially v-shaped portion 216 and tongue portion 217 extending from one end of the v-shaped portion 216. At the apex of the v-shaped portion 216, the pad 215 comprises a target sampling portion 218, which target sampling portion 218 has a sinusoidal shape, similar to that shown, in FIG. 1b, for example.

The first inner layer 209 includes a hole 219 which is slightly smaller than, and located directly over, the target sampling portion 218. The arrangement is such that, with the device 200 correctly located, with respect to the nose of a user, through appropriate use of the finger locators, when the user deposits a nasal sample between the wings 201, 202, the nasal sample may pass through the hole 219 and contact the sampling portion 218. Notably, even if the user were to deposit the sample on the second inner layer 210 of the wing 202 only, by virtue of closing the wings 201, 202 together, the sample may, nevertheless, contact, the sampling portion 218. To ensure that the sample may contact only the sampling portion 218 immediately after deposition, and not other elements of the device underneath the inner layers 209, 210, the inner layers 209, 210 may be formed of substantially fluid resistant material.

First and second lateral flow test strips 220, 221 are mounted on the backing layer 208 such as to be in fluid engagement with the pad 215. Once deposited on the target sampling portion 218 of sample pad 215, the device is configured such that the sample is transferrable by capillary action, from the target sampling portion 218 via a first arm 216a of the v-shaped portion 216, to a first end of each lateral flow test strip 220, 221 adjacent a head end 200a of the device 200. In this embodiment, the lateral flow test strips 220, 221 are conventional test strips, although other test strips or testing means applying the principles of immunochromatography or otherwise may be utilised in this or alternative embodiments. The first and second test strips 220, 221 may be considered to provide a test portion of the device 200.

Referring to FIG. 11, each test strip 220, 221 can include several zones that are arranged sequentially along the length of the strip, including a sample receiving zone 220a, a label-holding zone 220b, a test zone 220c, and a sink 220d. The zones may comprise chemically treated material, such as chemically treated nitrocellulose, located on a waterproof substrate. The design is such that the fluid sample, when transferred, from the sample pad 215 can continue to travel under capillary action through the sample receiving zone 220a, into the label-holding zone 220b, which contains a substance for labelling of a target, analyte, and into the test zone 220c where the sample will contact a test region or stripe 220e containing an immobilized compound capable of specifically binding the labelled target analyte or a complex that the analyte and labelling substance form. The presence of the labelled analyte in the sample generally results in a visually detectable colouring of the stripe 220e.

In addition to the test strip 220e, a control stripe 220f in the lest zone 220 can be provided to indicate that a testing procedure has been performed. The control stripe 220f can be located downstream of the test stripe 220e and is operable to bind and retain the labelling substance. Visible colouring of the control stripe 220f indicates the presence of the labelling substance resulting from the fluid sample flowing through test zone 220c. When the target analyte is not present in the sample, the test stripe 220e shows no visible colouring, but the accumulation of the label in control stripe 220f indicates that the sample has flown through test zone 220c. The sink (absorbent) zone 220d can then capture any excess sample. In this embodiment, the sample pad 215 is directly connected to the sample receiving zone 220a of each strip 220, 221, However, in other embodiments, the sample receiving zone 220a may be omitted, and the sample pad 218 may be configured to fluidly connect directly to the label-holding zone.

The test strips 220, 221 are arranged with their elongation directions configured substantially parallel to the fold lines 212, such that the strips can be enclosed by the elongate body of the spine 203 when the tacking layer 208 is folded along the fold lines 212. By enclosing the test strips 220, 221 in the spine 203, the strips, which can be relatively rigid and/or brittle in comparison to the pad 215, may be prevented from breaking. So that the user can see the control and capture lines 220e, 220f of the strips 220, 221 when the fold region 212 is enclosed by the spine 203, a window 222 is provided in the backing layer 208, and two windows 223, one for each test strip, are provided in the top plate 213. In this embodiment, the two test strips 220, 221 are configured to test for the presence of the influenza A and influenza B virus in the sample. However, in the present embodiment or other embodiments, testing for the presence of one of these viruses only, or testing of additional or alternative biological entities, is possible. The device 200 may be modified to include only one test strip, or to include more than two test strips.

The first and second test strips 220, 221 are located in a staggered arrangement, in particular, relative to the second test strip 221, the first test strip 220, which is located nearer to the pad 215 than the second test strip 221, is located inwardly from the edge of the backing layer 208 at the head end 200a of the device 200. The particular configuration is intended to ensure that the lengths of the fluid engagement, paths between the target portion 218 and the first and second test strips 220, 221 is substantially the same. Accordingly, during testing, sample can be expected to reach corresponding locations of the two strips 220, 221 at substantially the same time such that the results of testing indicated by the two test strips 220, 221 may be presented initially at substantially the same time. To bridge the additional gap between the first arm 216a and the first test strip 220, an inwardly extending projection 224 of the sample pad 215 is provided.

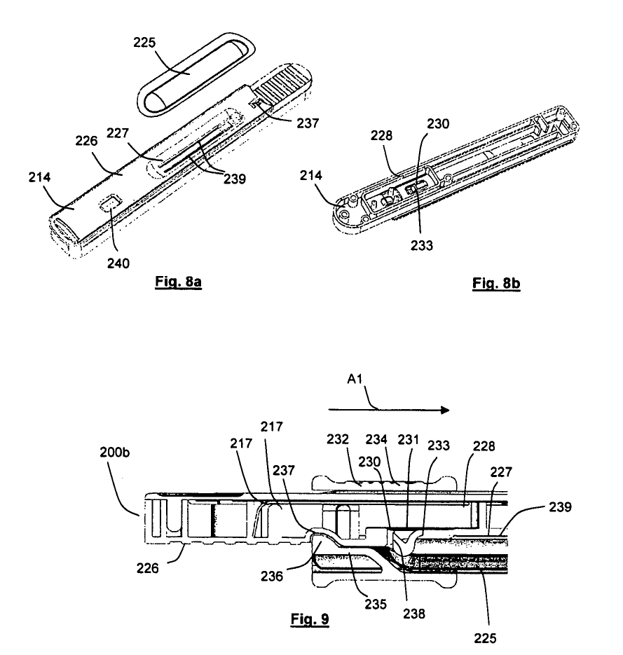

To assist in the transfer of the sample from the target portion 218 to the test strips 2201, 221, a liquid, e.g., a buffer solution is provided in the device 200. Initially, the liquid is sealed within a first reservoir. With reference to FIG. 8a, for example, the first reservoir is formed between a blister element 225 and a recess 227 in the bottom wall 226 of the main body 214 of the spine 203. The blister element 225 may be formed of Aclur™/polypropylene laminate, for example, and may be attached to bottom wall 226 of the main body via an adhesive. The first reservoir is arranged to hold the liquid underneath a second reservoir of the device 200, the second reservoir being empty of the liquid prior to use of the device 200. With reference to FIGS. 7 and 8b, for example, the second reservoir is formed from a substantially rectangular trough 228 at the top side of the main body 214 and a foil element 229 that seals the top of the trough 228.

In the bottom wall 226 of the main body 214, directly between the first and second reservoirs, an opening 230 is provided. The opening 230 is initially sealed by a pierceable film 231. The pierceable film 231 and opening 230 are designed such that, once the film 231 is pierced, liquid may travel from the first reservoir into the second reservoir. The tongue 217 of the pad 215 is configured to extend into the trough 228 of the second reservoir. Accordingly, when the liquid travels into the second reservoir, the liquid can be absorbed, over a period of time, by the tongue 217, whereupon the liquid will travel along the second arm 216b of the pad 215 to the target sampling portion 218 and combine with the deposited sample by travelling through the meandering flow path of the sampling portion 218. The combined sample and fluid will then travel along the first arm 216a of the pad 215 to the test strips 220, 221.

To pierce the film 231, an actuation mechanism is provided. The actuation element is intended to be operated after a sample has been deposited and the wings 201, 202 have been closed together. The actuation mechanism includes a slider 232, slidable along the elongation direction of the spine 203, and a piercing element 233, the piercing element projecting over the hole 230, adjacent the pierceable film 231. The slider 232 has a main body section 234, which is configured to partially surround the spine 203, and a flexible inner flange 235 extending from an inner surface of the main body section 234. The inner flange 235 has a projection 236 at its distal end, the projection 236 being biased by the flange 235 to press against the bottom wall 226 of the spine 203. The spine 203 may be considered to provide a track for controlled movement of the slider 232.

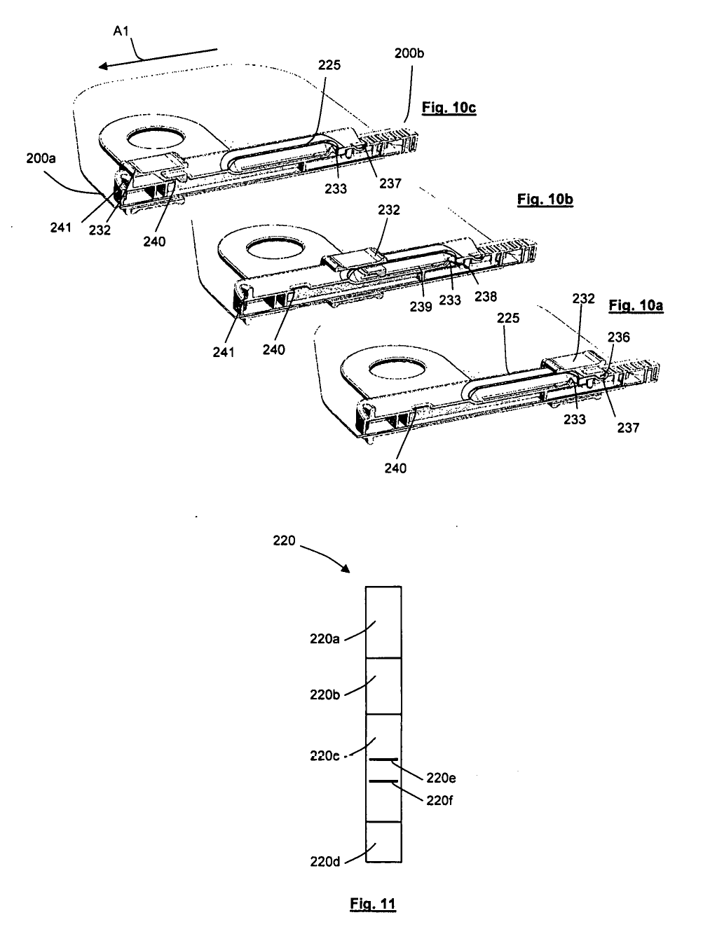

The operation of the actuation mechanism is now described in more detail with reference to FIGS. 9 and 10a to 10c. Referring to FIGS. 9 and 10a, prior to use the slider 232 is positioned, adjacent the tail end 200b of the device 200, with the projection 236 located in a first recess 237 in the bottom wall 226 of the main body 214 such as to prevent the slider 232 from moving freely relative to the spine 203. However, through the user pushing the slider 232 in the elongation direction of the spine 203, in a direction towards the head end 200a of the device, as indicated by arrow A1, the projection can be forced out of the recess 237, allowing the slider to move towards the blister element 225 of the first reservoir. The configuration of engagement surfaces between the projection 236 and recess 237, however, is such, as to pre vent the slider 232 from being moved in the opposite direction to direction A1.

With reference to FIG. 10b, once the slider 232 reaches the blister element 225, the projection 236 presses against the blister element 225, which element 225 in turn presses against the piercing element 233, forcing a sharp end 238 of the piercing element 233 against the pierceable film 231, causing the film 231 to break. The piercing element 233 is located towards the tail end of the first reservoir, and is therefore actuated almost immediately upon the contact between the projection 236 and the blister element 225. As the slider 232 continues to move in the same direction A1, the projection 236 effectively inverts the blister element 225 towards the bottom of the recess 227, forcing liquid from the first reservoir into the trough 228 of the second reservoir, via the opening 230 (the inversion is not represented in FIG. 10b). Once the film is broken, to ensure that the liquid is not prevented from moving towards the opening 230 by opposing movement of the projection 236 across the blister element 225, which might otherwise invoke a seal between the inverted blister element 225 and the bottom of the recess 227, one or more fluid channels 239 are provided in the bottom surface of the recess 227. The channels 239 ensure that the solution can travel underneath the projection and inverted blister element 225, towards the opening 230.

With reference to FIG. 10c, once the slider 232 passes over the blister element 225, the slider 232 is arranged take up a rest position adjacent the head end 200a of the device 200. To maintain the slider 232 in this position, preventing it from moving freely relatively to the spine 203, the projection 236 is arranged to seat in a second recess 240 and the head end of the slider 200 is arranged to abut a stop element 241 at the head end of the spine 103 such that the slider 232 is prevented from sliding off the spine 203. The configuration of engagement surfaces between the projection 236 and recess 240 is such as to prevent the slider 232 from being returned to the tail end 200b of the device 200. Accordingly, since the slider 232 will be maintained at the head end 200a of the device, it can remain immediately apparent to the user that the device 200 has been used, reducing the likelihood of an attempted re-use of the device 200.

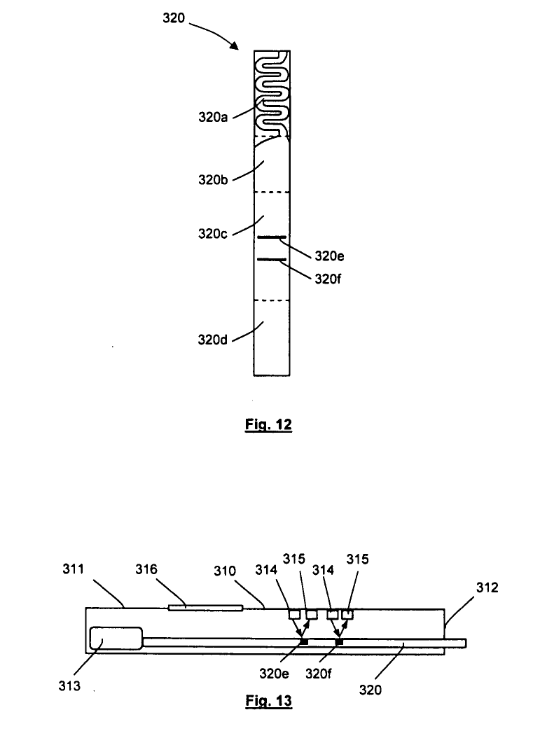

A test device 300 according to another embodiment of the present disclosure is now discussed with reference to FIGS. 12 and 13.

The test device 300 includes a lateral flow test strip 320 configured in a similar manner to the lateral flow test strip 220 described above with reference to FIG. 11. Again, the test strip 320 includes several zones that are arranged sequentially along the length of the strip, including a sample receiving zone 320a, a label-holding zone 320b, a test zone 320c, including a test stripe 320e and a control stripe 320f, and a sink 320d. The zones may comprise chemically treated absorbent material such as chemically treated nitrocellulose, and are located on a waterproof substrate. However, in contrast to the lateral flow test strip 220 of the preceding embodiment, the test strip 320 includes absorbent material that, at the sample receiving zone 320a, defines a meandering flow path.

The test device 300 includes an electronic read apparatus 310. The read apparatus 310 includes a housing 311 with an opening 312 at one end though which the test strip 320, after receiving a sample, can be pushed into the housing 311 to a position at which it fluidly engages a reservoir 313 contained in the housing. Buffer solution contained in the reservoir 313 can travel along the meandering flow path of the sample receiving zone 320a, where the solution combines with deposited sample and then travels through subsequent zones 320b, 320c, 320d of the lateral flow test device, ultimately leading to a colour change at one or both of the test and control stripes 320e, 320f.

Read circuitry in the test device 300 includes LEDs 314 that illuminate the stripes 320e, 320f and photodetectors 315 that determine the amount of light reflected from the stripes 320e, 320f. A processor is configured to determine whether a biological entity is present, in the sample based on the amount of reflected light detected by the photodetectors 315 and configured to display the results of testing on an electronic screen 316.

It will be appreciated by persons skilled in the art that numerous variations and/or modifications may be made to the above described embodiments, without departing from the broad general scope of the present disclosure. The present embodiments are, therefore, to be considered in all respects as illustrative and not restrictive.

1. A test device for determining the presence or absence of a biological entity in a biological sample from a human or animal body, the test device comprising a sampling portion, the sampling portion comprising absorbent material forming a liquid flow path having a series of bends.

2. The test device of claim 1, wherein the series of bends includes a plurality of bends of at least 90 degrees.

3. The test device of claim 1, wherein the series of bends includes a plurality of bends of approximately 180 degrees.

4. The test device of claim 1, wherein the sampling portion is adapted to receive the sample directly from the human or animal at positions of the sampling portion including at a bend of the liquid flow path.

5. The test device of claim 1, wherein the flow path has a meander shape.

6. Test device of claim 1, wherein the flow path has a substantially sinusoidal shape.

7. The test device of claim 1, wherein the flow path has a substantially square or rectangular wave shape.

8. The test device of claim 1, wherein the flow path comprises a plurality of substantially straight sections connected to each other via one or more bend sections, wherein the straight sections are substantially parallel to each other and adjacent straight sections are separated from each other by a gap.

9. The test device of claim 8, wherein the gap separating adjacent straight sections is from about 0.5 mm to 5 mm.

10. The test device of claim 8, wherein the gap separating adjacent straight sections is from 1 mm to 3 mm.

11. The test device of claim 8, wherein the gap separating adjacent straight sections is about 2 mm.

12. The test device of claim 8, wherein the gaps are absent of any material.

13. The test device of claim 1, wherein the flow path is defined by opposing outer edges of the sampling portion.

14. The test device of claim 1, wherein the flow path is defined by a first material of the sampling portion, the first material being adjacent a second material of the sampling portion, the second material being less absorbent than the first material.

15. The test device of claim 1, wherein the sampling portion comprises a sample receiving surface adapted to receive a sample deposited thereon and a liquid transfer portion that is connected or is configured to be connected to the sampling portion such that liquid is transferable from the liquid transfer portion to the flow path to combine with sample deposited on the receiving surface by passing through the flow path.

16. The test device of claim comprising a reservoir connected to the liquid transfer portion.

17. The test device of claim 1, wherein the test device comprises a test portion connected to the sampling portion and wherein the test device is configured such that liquid combined with the sample at the flow path is transferred to the test portion by capillary action.

18. The test device of claim 17, wherein the test device is a lateral flow test device and the test portion is configured to test for the presence or absence of a biological entity in the sample using immunochromatography.
